(19) United States
(12) Patent Application Publication
(10) Pub. No.: US 2009/0301484 A1
(43) Pub. Date: Dec. 10, 2009

(54) METHOD AND APPARATUS FOR DELIVERING A FLUID TO A PATIENT

(76) Inventor: Colin Dunlop, East Ryde (AU)

Correspondence Address: KNOBBE MARTENS OLSON & BEAR LLP, 2040 MAIN STREET, FOURTEENTH FLOOR, IRVINE, CA, 92614, US

(21) Appl. No.: 12/483,959

(22) Filed: Jun. 12, 2009

Related U.S. Application Data
(63) Continuation of application No. PCT/AU2007/001920, filed on Dec. 13, 2007.

(30) Foreign Application Priority Data
Dec. 13, 2006 (AU) 2006252044

(51) Int. Cl. A61M 16/01

(52) U.S. Cl. 128/203.12

(57) ABSTRACT

The present invention relates to a method and apparatus for delivering medicated fluid or anaesthetic to a patient. An issue, particularly with small patients, is the issue of dead space in an anaesthetic circuit which can lead to hypoxia and difficulty with achieving anaesthesia. An apparatus in accordance with the present invention minimises or eliminates dead space by introducing fresh gas (e.g. containing anaesthetic) proximal to a respiratory opening of the subject's respiratory tract, and inducing a fluid flow to reduce re-breathing of fluid exhausted from the respiratory tract.

### CROSS-REFERENCE TO RELATED APPLICATIONS

This application is a continuation of International Application No. PCT/AU2007/001920, filed Dec. 13, 2007, which claims the benefit of Australian Patent Application No. 2006252044, filed Dec. 13, 2006, both of which are incorporated herein by reference in their entirety.

### BACKGROUND

1. Field

The present invention relates to a method and apparatus for delivering fluid to a patient, and, particularly, but not exclusively, to a method and apparatus for delivering a medicated fluid or anaesthetic to a patient.

2. Description of the Related Technology

In anaesthesia, anaesthetic gases are delivered to patients usually utilising one of a number of types of anaesthetic circuit. The anaesthetic circuit is arranged to deliver a regulated dose of anaesthetic to the patient, along with oxygen or a gas mixture including oxygen or atmospheric air. It is important that the patient be supplied with and breathes a correct mixture of oxygen and anaesthetic gas. Too little oxygen reaching the lungs, or too much re-breathed CO2 reaching the lungs, can lead to significant problems, including hypoxia and death.

A common feature of anaesthetic circuits is the connection between the circuit (containing anaesthetic gas) and the patient. This connection is most commonly either via a tube placed through the mouth or nose into the airway (endotracheal tube) or a face mask sealed to the face. A mask delivers gases to a respiratory opening of the patient for inspiration and to receive expired gases for removal from the patient.

An important issue with anaesthetic circuits is the issue of “dead space”. This is the volume of gas at expiration that the patient must re-breathe before getting gas that does not contain carbon dioxide. The dead space includes the patient's own air passageway dead space (e.g. trachea, or cavity) and, with anaesthetic circuits, a certain amount of “machine dead space”.

The more machine dead space the more volume a patient must breathe in before they breathe gas that does not contain carbon dioxide. For adult humans and large animals, the amount of machine dead space is often not large compared to the anatomic dead space and the total volume of their airways. For small animals and small humans, however, the amount of machine dead space can become critical. For example, a two kilogram animal, may have a total tidal volume of 20-30 mls. A third of this volume will be anatomic dead space. Any additional machine dead space may cause significant issues for anaesthesia. For example, an extra 10 mls of dead space can cause the animal to hyperventilate, become hypoxic and die.

Even if patients do not become hypoxic, the delivery of anaesthetic is much less efficient and it is more difficult to achieve anaesthesia.

To address machine dead space issues, it is common to intubate patients (introducing a tube into the trachea). For small animals and infants, however, this is not always a satisfactory solution. With smaller airways, the diameter of the tube lumen becomes so small that the patient may not be able to draw enough gas into their lungs (the small diameter tubing provides significant resistance to inspiration).

Small, tight fitting masks have been designed for small animals and infants. Nevertheless, even small masks still add dead space which can be significant.

The delivery of fluids to patients is an important aspect of medical care. Pharmaceuticals may often be delivered as a vapour or a gas to a patient's airway, for example. Examples include pharmaceuticals for treatment of asthma. It may be important that a precise dose of medicated gas be delivered. With asthma pharmaceuticals, a delivery device including a gas canister and “puffer” is often utilised. In such devices more gas than necessary may be delivered, leading to either inaccuracy of the inspired concentration or excessive waste.

### SUMMARY

In accordance with a first aspect, the present invention provides a method of delivering a fluid to the respiratory tract of a human or animal subject, comprising the steps of introducing fresh fluid proximal to a respiratory opening of the subject, and inducing a fluid flow to reduce re-breathing of fluid exhausted from the respiratory tract, the step of inducing a fluid flow comprising the step of exhausting exhaled fluid from a position distal to the proximal position of introduction of fresh fluid.

In an embodiment, the fluid is introduced into and exhausted out of an enclosed space by the respiratory opening.

In an embodiment, the respiratory opening is the nose or mouth of the subject, or nose and mouth combined.

In an embodiment, the enclosed space is formed by a mask.

In an embodiment, the fluid is anaesthetic. In an embodiment where there is an enclosed space, the enclosed space is part of an anaesthetic circuit.

In an embodiment, the fluid is a medicinal fluid.

In an embodiment, the subject may be a animal or human of 45 kilograms or less, in an embodiment 35 kilograms or less, in an embodiment 25 kilograms or less, in an embodiment 15 kilograms or less, in an embodiment 10 kilograms or less, in an embodiment 5 kilograms or less, and in an embodiment 2 kilograms or less.

It is an advantage of at least an embodiment of the invention that fresh fluid is introduced in such a way as to reduce or avoid re-breathing of fluid exhausted from the respiratory tract. In anaesthesia, this may have the advantage of effectively reducing machine dead space. The expirate is removed from the opening of the respiratory tract by the fluid flow. This embodiment is therefore particularly suited to anaesthesia of small humans and animals, as it reduces machine dead space to reduce re-breathing of expired carbon dioxide.

The ability to induce a fluid flow and introduce fresh fluid proximal to a respiratory opening of a subject also may have advantages for introduction of precise doses of other medicinal fluids to subjects. For example, an immediate concentration of medicine may be introduced to the patient's airway utilising this method.

In accordance with a second aspect, the present invention provides an apparatus for facilitating delivery of a fluid to a respiratory tract of a human or animal subject, the apparatus comprising an enclosure having a opening arranged to fit over a respiratory opening of the subject, an inlet arranged to enable the introduction of fresh fluid proximal to the respiratory opening, and a fluid flow inducing mechanism arranged to induce a fluid flow to reduce re-breathing of fluid exhausted from the respiratory tract, comprising an outlet from the enclosure, positioned distally with respect to the inlet.

In an embodiment, the enclosure is a mask for covering the nose or mouth of the subject, or nose and mouth of the subject.

In an embodiment, the apparatus is arranged to be connected to an anaesthetic circuit for the delivery of anaesthetic. In an embodiment, the enclosure may be incorporated into the anaesthetic circuit.

In an embodiment, the enclosure may be arranged to be attached to a delivery device for delivering a medicinal fluid to the subject. The enclosure may be integrated with the delivery device. In an embodiment, the medicinal fluid may be a fluid for the treatment of asthma.

In accordance with a third aspect, the present invention provides an anaesthetic circuit comprising an apparatus in accordance with the second aspect of the invention.

In accordance with a fourth aspect, the present invention provides an apparatus for the delivery of a fluid to the respiratory tract of a human or animal subject, the apparatus including an enclosure having an opening arranged to fit over a respiratory opening of the respiratory tract, an inlet for introducing fresh fluid proximal to the opening and an outlet distal to the inlet for exhausting expired fluid.

In an embodiment, the enclosure is a mask.

In an embodiment, the enclosure is arranged to fit over the nose or mouth of the subject, or nose and mouth.

In accordance with a fifth aspect, the present invention provides a method of delivering a fluid to the respiratory tract of a human or animal subject, comprising the steps of introducing fresh fluid to a respiratory opening of the respiratory tract by inducing a fluid flow to reduce re-breathing of fluid exhausted from the respiratory tract, the fluid flow being induced by inducing a positive pressure for the fluid flow by the respiratory tract.

In accordance with a sixth aspect, the present invention provides an apparatus for facilitating delivery of a fluid to a respiratory tract of a human or animal subject, the apparatus comprising an enclosure having an opening arranged to fit over the respiratory opening of the subject, an inlet arranged to enable the introduction of fresh fluid and an outlet for allowing exhaustion of the fluid, a fluid flow being induced to reduce re-breathing of fluid exhausted from the respiratory tract, the fluid flow being induced by providing a positive pressure at the inlet with respect to the outlet.

### BRIEF DESCRIPTION OF THE DRAWINGS

Features and advantages of the present invention will become apparent from the following description of embodiments thereof, by way of example only, with reference to the accompanying drawings, in which:

FIG. 1 is a diagram of a prior art anaesthetic circuit for the delivery of anaesthetic to a subject;

FIG. 2 is a diagram of an alternative prior art anaesthetic circuit;

FIG. 3 is a diagram of an anaesthetic circuit including an apparatus in accordance with an embodiment of the present invention;

FIG. 4 is a drawing of an apparatus in accordance with an embodiment of the present invention;

FIG. 5 is a side view of an apparatus in accordance with an embodiment of the present invention;

FIG. 6 is a back view of the embodiment of FIG. 5;

FIG. 7 is an underneath view of the embodiment of FIG. 5;

FIG. 8 is a cross sectional view of the embodiment of FIG. 5, and

FIG. 9 is a perspective view on a smaller scale of the embodiment of FIG. 5.

### DETAILED DESCRIPTION OF EMBODIMENTS

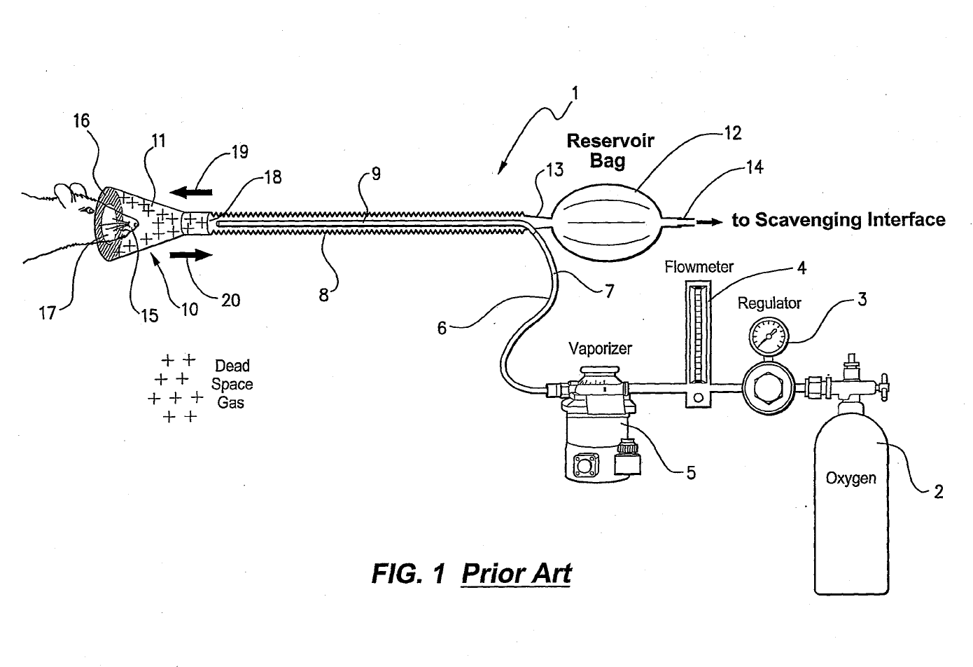

Referring to FIG. 1, a prior art anaesthetic circuit of the non-re-breathing type is shown. Such non-re-breathing circuits are often used to anaesthetise animals, particularly small animals, and also children and infant human subjects. The anaesthetic circuit is designated generally be reference numeral 1.

The anaesthetic circuit 1 includes a means of providing oxygen, in this case being a gas cylinder 2 containing oxygen. A regulator 3 is provided to regulate the pressure of the oxygen supply and a flow meter 4 provides an indication of the gas flow. A vaporiser 5 is provided for the introduction of anaesthetic (and perhaps other medicinal gases) to the gas flow. A tube 6 having a central lumen 7 extends from the vaporiser into a further tube 8 providing a further lumen 9 surrounding the tube 6. A mask 10 is provided connected to corrugated tube 8. The lumens 7 of the tube 6 and 9 of the corrugated tube 8 open into an interior space 11 of the mask 10. A reservoir bag 12 is provided connected to the distal end 13 of the corrugated tube 8 and has outlet 14 which proceeds to a Scavenging Interface (not shown).

The apparatus of FIG. 1 is a standard type of non-re-breathing anaesthetic circuit which is well known and, with variations, finds wide application.

In this prior art circuit, the mask shown 10 is of the type which is mainly used for relatively small animals (under 30 kilograms and even under 10 kilograms and 5 kilograms). The mask 10 forms an enclosure which encloses an opening to the respiratory tract of the animal, in this case it fits over the nose and the mouth, referenced by reference numeral 15. A flexible gasket 16 borders a forward opening 17 of the mask 10 to provide tight seal of the enclosure 11 with the nose/mouth of the subject. The mask has a single opening 18 at a part of the mask distal from the opening 16, the opening 18 arranged to allow the passage of inspired and expired gases (indicated by arrow 19 and arrow 20, respectively).

In operation, fresh gas including anaesthetic (and any other required medicated gases) is introduced by line 6 into the space 11 within the mask 10, via opening 18. Exhaled gases travel via the opening 18 and a lumen 9 of the corrugated tube 8 to the Scavaging Interface.

Dead space 11 within the mask 10 comprises anaesthetic dead space. Dead space constitutes extra volume that the subject must inspire before they inspire fresh gas. Particularly for small animals and small humans, the volume 11 in this dead space can become critical. For example, for a small subject having a small tidal volume, any additional machine volume can significantly affect the composition of the gas being breathed using these types of circuits. Hyperventilation and hypoxia are common problems. Also a common problem, is difficulty in inducing anaesthesia because of the inability to deliver the required dosage of anaesthesia.

Attempts have been made to overcome this problem by designing very small volume masks in order to reduce the dead space to as little as possible. These small volume masks have to fit tightly and are often very uncomfortable and cause stress to the subject. Further, different ranges of different sizes of masks need to be designed for different sizes of subject.

Even where masks are designed to minimise volume, there is still sufficient machine dead space to cause the problems discussed above.

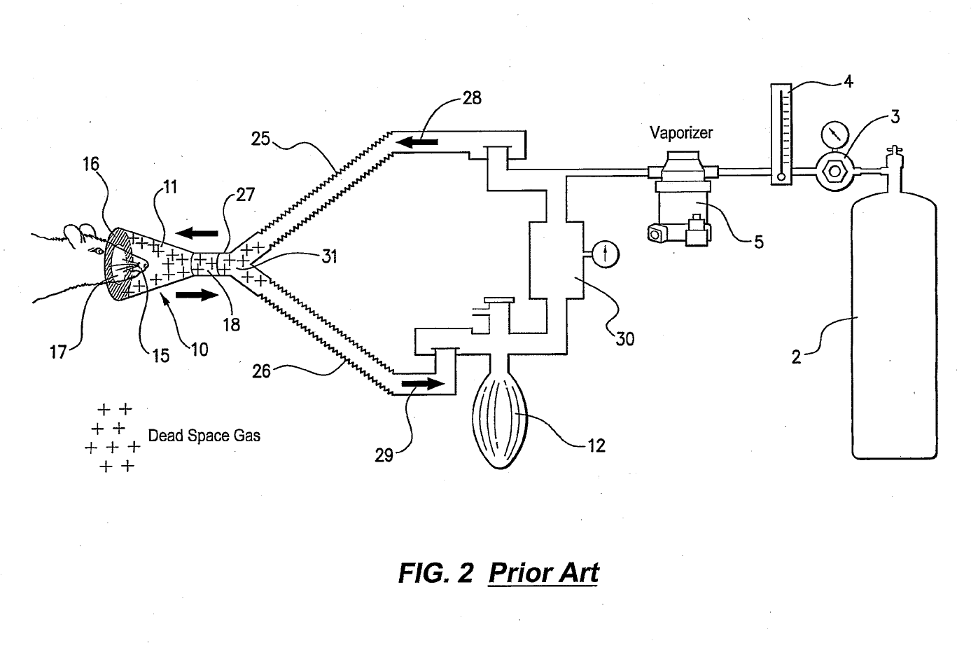

Referring to FIG. 2, an alternative prior art anaesthetic circuit is illustrated. This is a re-breathing type circuit where oxygen/air may be re-breathed after it has been scrubbed of carbon dioxide. Components which are the same as the components of the FIG. 1 system have been given the same reference numerals and no further description will be given. The system includes anaesthesia lines 25 & 26 which form a “Y” connection 27 with the distal opening 18 of the mask 10. Fresh gas flow comes down line 25 from the vaporiser 5, in the direction of arrow 8. Exhaust gas flow travels up the other arm of the “Y”, line 26, in the direction of arrow 29. Scrubber 30 removes carbon dioxide from the exhaust gas and it re-enters the line 25 at the other side of the scrubber 30.

With this type of circuit there is even more machine dead space. As well as the dead space volume in enclosure 11 of the mask, there is also an extra dead space volume c where the arms 25, 26 of the “Y” connect, being volume 31 distal to the opening 18 of the mask 10.

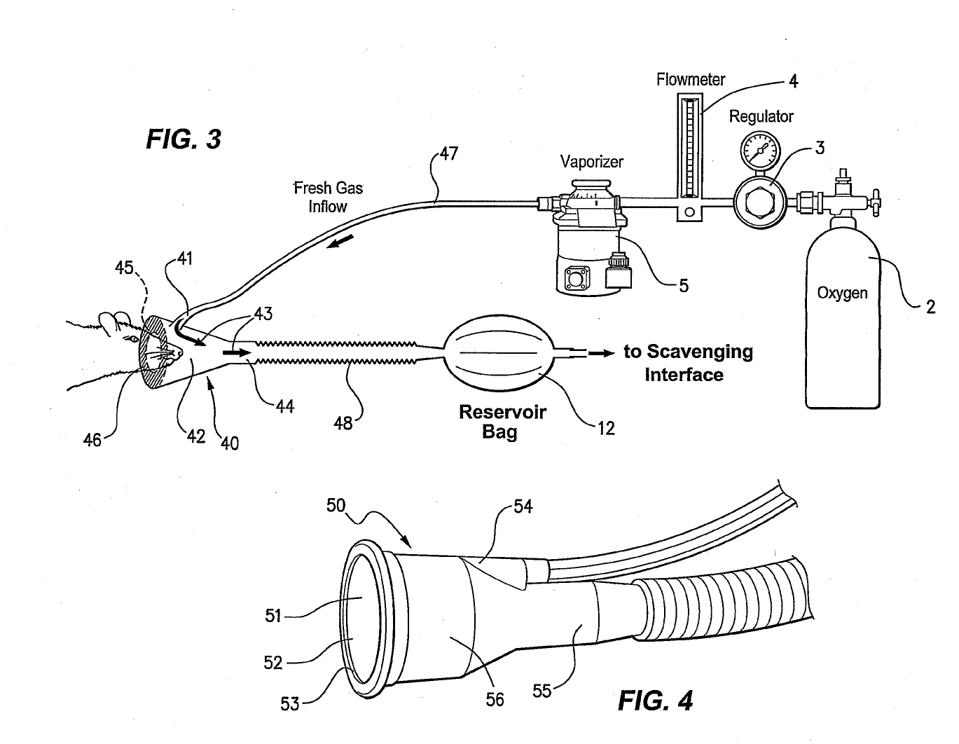

An embodiment of the present invention will now be described with reference to FIG. 3.

An apparatus in accordance with an embodiment of the present invention is designated by reference numeral 40. The apparatus includes an inlet 41 for introducing fluid proximal to a respiratory opening 42 of a human or animal subject, and is arranged to facilitate the induction of a fluid flow which operates to reduce re-breathing of fluid exhausted from the respiratory opening 42. In this embodiment the induced flow is uni-directional, past the respiratory opening 42, as indicated by arrows 43, and out of a further opening, outlet 44 from the apparatus 40, the outlet 44 being distal to the proximal inlet 41.

The induced fluid flow 43 results in what otherwise might have been dead space within the apparatus 40 being eliminated or reduced for the purposes of the subject breathing in fluid.

In more detail, in this embodiment, the apparatus 40 is a mask including an opening 45 for receiving the subject's nose or mouth, or nose and mouth and also including a gasket 46 to provide at least a loose seal about the subject. Note that the gasket 45 may not be required in some cases.

The mask 40 includes the inlet 41 which is proximal to the opening 45 and an outlet 44 which is distal from the proximal inlet. The outlet 44 together with the inlet 41 provides a pathway for gas flow impelled by positive pressure at the inlet 41 compared to the outlet 44.

In this embodiment, the apparatus 40 is shown connected to a length of corrugated tubing then a re-breathing bag and scavenging interface. Instead of the position in the prior art where the fresh gas enters through the distal opening of the mask, however, in this arrangement fresh gas enters via the proximal inlet 41 and exhaust gas is expired through the distal outlet 44.

Some components of the anaesthetic circuit have been given the same reference numerals as the components of FIGS. 1 & 2. these components are similar and no further description will given. A fresh gas flow tube 47 extends from the vaporiser 5 to the inlet 41 of the mask 40. A corrugated outlet tube 48 is connected between the outlet 44 of the mask 40 and the reservoir bag 12.

Note that the mask may be separate from the anaesthetic circuit, having connections for the inlet and the outlet 44. Alternatively, it may be integrated with components of the anaesthetic circuit (e.g. inlet line 47 and outlet line 48).

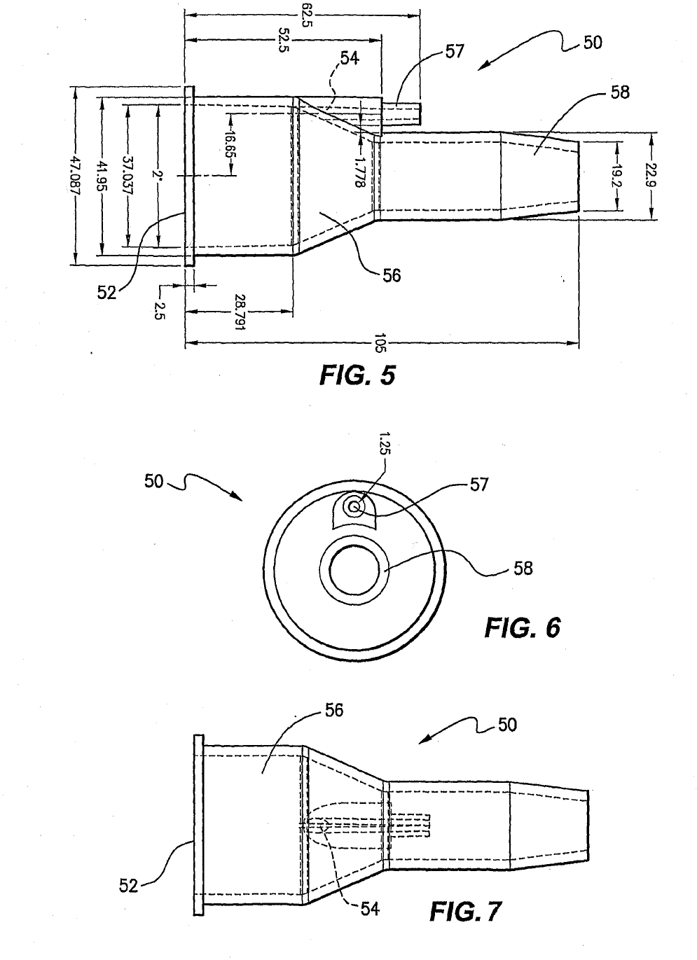

FIGS. 4 through 9 show in more detail an embodiment of an apparatus in accordance with the present invention. This embodiment is a mask for the delivery of fluid to a subject. Dimensions shown in the drawings are example dimensions only. The mask may be dimensioned and shaped differently.

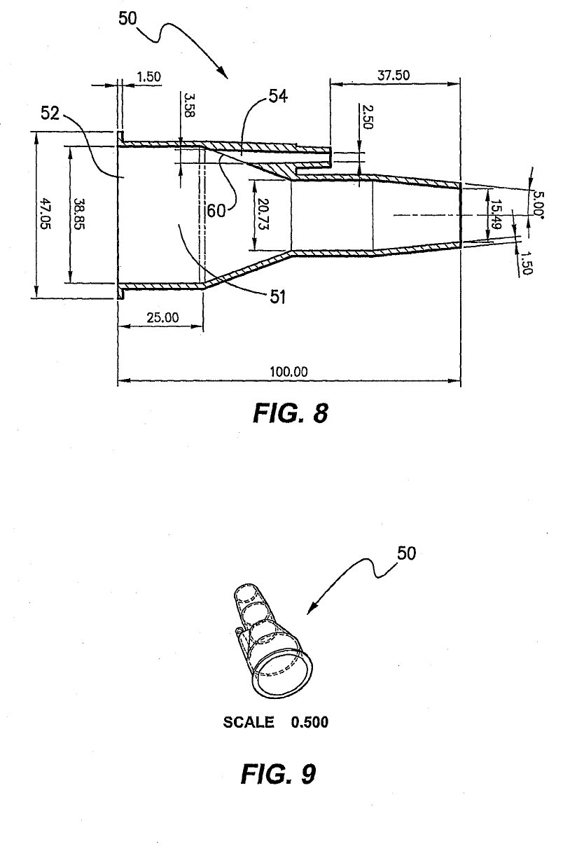

The mask 50 defines an enclosure 51 and includes an opening 52 which is arranged to be placed about or over a respiratory opening of a subject. The opening 52 includes a resilient gasket 53. The mask 50 includes an inlet 54 which opens into the enclosure 51 proximal to the opening 52 and in use adjacent to the respiratory opening of the subject. The mask also includes an outlet 55 which is positioned distally of the inlet 54. The mask 50 includes a wall 56 bounding the enclosure 51. The inlet 54 includes a connector piece 57, which may be used to connect to an inlet tube of an anaesthetic circuit, or to another device for providing fluid. The outlet 55 also includes a connector 58 which may be used to connect to the exhaust line of an anaesthetic circuit. From FIG. 8 it can be seen clearly that the proximal end 60 of the inlet 54 opens into the enclosed space 51 proximal to the opening 52.

The mask 50 can in fact be considered as an integrated anaesthetic circuit in its own right. For small patients, for example, any extra line (e.g. corrugated tubing) on the outlet 55 may not be required. A re-breathing bag and a scavenging circuit could be connected directly to the connector 58. This limb (55, 58) of the mask in fact constitutes an expired limb of the circuit integrated into the apparatus. In embodiments, this limb can be as long and with as much volume as required—the tube can be made a metre long, for example. Because of the operation of the apparatus, the volume of this limb does not add to the dead space of the machine. The limb can even be made flexible.

The apparatus and method of the present invention is not limited to use in anaesthesia. It may also be used for the dispensing of other medicinal fluids, such as pharmaceuticals for treating asthma, for example. The apparatus and method preferably have the advantage that they enable the delivery of an immediate concentration of medicine to the respiratory tract of a subject.

In anaesthesia, the apparatus and method have particular advantage in small human and animal subjects. Slight positive pressure of gas at the inlet may be required in order to induce unidirectional flow. For a 5 kilogram animal for example, the flow rate may be in order of 3 litres per minute and for a 10 kilogram animal or human in the order of 6 litres per minute. For larger animals the flow rate will need to be greater but, even where there is a lower flow rate, it will still result in at least a reduction of dead space. Significant advantage will be gained as long as the flow rate is higher than the minute ventilation of the subject.

A further advantage with small animals and small human patients is that it not necessary to make a range of sizes of masks to fit different sizes of patients. One or a few different sizes of masks may be suitable, as dead space is not as big an issue with embodiments of the present invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

What is claimed is:

1. A method of delivering a fluid to the respiratory tract of a human or animal subject, comprising the steps of introducing fresh fluid proximal to a respiratory opening of the respiratory tract, and inducing a fluid flow to reduce re-breathing of fluid exhausted from the respiratory tract, the step of inducing a fluid flow comprising the step of exhausting exhaled fluid from a position distal to the proximal position of introduction of fresh fluid.

2. A method in accordance with claim 1, wherein the fluid is introduced into and exhausted out of an enclosed space by the respiratory opening.

3. A method in accordance with claim 2, wherein the enclosed space is formed by a mask.

4. A method in accordance with claim 1, wherein the fluid is anaesthetic.

5. A method in accordance with claim 1, wherein the fluid is a medicinal fluid.

6. An apparatus for facilitating delivery of a fluid to a respiratory tract of a human or animal subject, the apparatus comprising an enclosure having an opening arranged to fit over a respiratory opening of the subject, an inlet arranged to enable the introduction of fresh fluid proximal to the respiratory opening, and a fluid flow inducing mechanism arranged to induce a fluid flow to reduce re-breathing of fluid exhausted from the respiratory tract, comprising an outlet from the enclosure, positioned distally with respect to the inlet.

7. An apparatus in accordance with claim 6, wherein the enclosure is a mask.

8. An apparatus in accordance with claim 6, wherein the enclosure is arranged to be connected to an anaesthetic circuit for the delivery of anaesthetic.

9. An apparatus in accordance with claim 6, wherein the enclosure is incorporated into an anaesthetic circuit for the delivery of anaesthetic.

10. An apparatus in accordance with claim 6, the enclosure being arranged to be attached to a delivery device for delivery of a medicinal fluid to the subject.

11. An apparatus in accordance with claim 6, the enclosure being integral with a delivery device for delivering a medicinal fluid to the subject.

12. An apparatus in accordance with claim 10, wherein the medicinal fluid is a fluid for the treatment of asthma.

13. An anaesthetic circuit comprising an apparatus in accordance with claim 6.

14. An apparatus for the delivery of a fluid to the respiratory tract of a human or animal subject, the apparatus including an enclosure having an opening arranged to fit over a respiratory opening of the respiratory tract, an inlet for introducing fresh fluid proximal to the opening and an outlet distal to the inlet for exhausting expired fluid.

15. An apparatus in accordance with claim 14, when the enclosure is a mask.

16. A method of delivering a fluid to the respiratory tract of a human or animal subject, comprising the steps of introducing fresh fluid to a respiratory opening of the respiratory tract by inducing a fluid flow to reduce re-breathing of fluid exhausted from the respiratory tract, the fluid flow being induced by inducing a positive pressure for the fluid flow by the respiratory tract.

17. A method in accordance with claim 16, wherein the fluid is introduced into an enclosed space by the respiratory opening, the fluid being introduced via an inlet to the enclosure at a positive pressure with respect to an outlet of the enclosure from which the fluid is exhausted.

18. A method in accordance with claim 17, wherein the positive pressure is induced at least partly by the inlet for the fluid being of smaller dimension than the outlet for the fluid.

19. An apparatus for facilitating delivery of a fluid to a respiratory tract of a human or animal subject, the apparatus comprising an enclosure having an opening arranged to fit over the respiratory opening of the subject, an inlet arranged to enable the introduction of fresh fluid and an outlet for allowing exhaustion of the fluid, a fluid flow being induced to reduce re-breathing of fluid exhausted from the respiratory tract, the fluid flow being induced by providing a positive pressure at the inlet with respect to the outlet.

20. An apparatus in accordance with claim 19, wherein the positive pressure is at least partly induced by the inlet being smaller in dimension than the outlet.
